(19) United States
(12) Patent Application Publication
(10) Pub. No.: US 2016/0174870 A1
(43) Pub. Date: Jun. 23, 2016

(54) APPARATUS FOR MEASURING BODY WEIGHT AND METHOD OF MEASURING BODY WEIGHT BY USING THE SAME

(71) Applicant: SAMSUNG ELECTRONICS CO., LTD., Suwon-si (KR)

(72) Inventors: Yeolho LEE, Anyang-si (KR); Kak NAMKOONG, Seoul (KR); Kunsun EOM, Seoul (KR); Myounghoon JUNG, Bucheon-si (KR); Seongho CHO, Gwacheon-si (KR)

(73) Assignee: SAMSUNG ELECTRONICS CO., LTD., Suwon-si (KR)

(21) Appl. No.: 14/809,346

(22) Filed: Jul. 27, 2015

(30) Foreign Application Priority Data
Dec. 23, 2014 (KR) 10-2014-0187506

(51) Int. Cl. A61B 5/053; G01G 19/50; A61B 5/00

(52) U.S. Cl. CPC A61B 5/0537; A61B 5/4872; A61B 5/681; G01G 19/50

(57) ABSTRACT

A body weight measuring apparatus and a body weight measuring method are provided. The body weight measuring apparatus includes an input unit configured to receive body information of a user other than bio impedance information; a bio impedance obtaining unit comprising a plurality of electrodes configured to be in contact with the user to measure a bio impedance of the user; and a body weight obtaining unit configured to obtain a weight of the user based on the measured bio impedance and the received body information.

### CROSS-REFERENCE TO RELATED APPLICATION

This application claims priority from Korean Patent Application No. 10-2014-0187506, filed on Dec. 23, 2014 in the Korean Intellectual Property Office, the disclosure of which is incorporated herein in its entirety by reference.

### BACKGROUND

1. Field

Apparatuses and methods consistent with exemplary embodiments relate to an apparatus for measuring a body weight and a method of measuring a body weight by using the same.

2. Description of the Related Art

With medical development and increase in the average life span, interest in health management is increasing. Therefore, interest in medical devices such as small and medium medical devices provided in public institutions or small medical devices and health care apparatuses that may be worn by individuals as well as various medical devices used in hospitals or health centers is increasing.

A body weight measuring apparatus, which is a kind of health care apparatus, measures the weight of a body to be inspected by using gravity. The body weight measuring apparatus may measure the weight of the body to be inspected by using gravity only when the body to be inspected is positioned on the body weight measuring apparatus. Therefore, the body weight measuring apparatus using gravity may not be used in a place with many limitations or by a patient whose body may not be positioned on the body weight measuring apparatus.

### SUMMARY

One or more exemplary embodiments provide a body weight measuring apparatus without spatial limitations and a body weight measuring method by using the same.

Further, one or more exemplary embodiments provide a computer-readable recording medium in which a program for executing the method by a computer is recorded.

According to an aspect of an exemplary embodiment, there is provided a body weight measuring method performed by a body weight measuring apparatus. The method may include: receiving body information of a user other than bio impedance information by an input unit of the apparatus; measuring bio impedance of the user by a bio impedance obtaining unit of the apparatus; and obtaining, by a processor of the apparatus, a weight of the user based on the measured bio impedance and the received body information.

The bio information may include a height of the user, and the obtaining the weight of the user may include calculating the weight in accordance with the body information and a magnitude of (the height of the user)²/(the bio impedance).

The body information may further include at least one of an age and a gender.

The bio information may include a height of the user, and the obtaining the weight of the user may include calculating the weight in accordance with the body information and a magnitude of (the height of the user)/(the bio impedance).

The body information may further include at least one of an age and a gender.

The obtaining the weight of the user may include calculating the weight in accordance with the body information and a magnitude of the bio impedance.

The body information may include at least one of an age, a gender, and a height of the user.

The measuring the bio impedance may include measuring the bio impedance based on a 4-point measuring method or a 2-point measuring method.

The measuring the bio impedance based on the 4-point measuring method may include applying a constant current to two electrodes among four electrodes of the apparatus and measuring the bio impedance through the other two electrodes among the four electrodes.

The measuring the bio impedance based on the 2-point measuring method may include connecting first and second electrodes of four electrodes of the apparatus so that the first and second electrodes are switched into an input electrode of the apparatus and connecting third and fourth electrodes of the four electrodes so that the third and fourth electrodes are switched into an output electrode of the apparatus.

According to another aspect of an exemplary embodiment, there is provided a non-transitory computer-readable storage medium storing a program that is executable by a computer to perform the body weight measuring method.

According to another aspect of an exemplary embodiment, there is provided a body weight measuring apparatus including: an input unit configured to receive body information of a user other than bio impedance information; a bio impedance obtaining unit comprising a plurality of electrodes configured to be in contact with the user to measure a bio impedance of the user; and a body weight obtaining unit configured to obtain a weight of the user based on the measured bio impedance and the received body information.

The body weight measuring apparatus may further include: a memory configured to store a program that calculates the weight of the user; and a processor configured to load the program and calculate the weight of the user in accordance with the body information and a magnitude of the bio impedance by using the program.

The bio impedance obtaining unit may be further configured to measure the bio impedance based on a 4-point measuring method or a 2-point measuring method.

The plurality of electrodes may include four electrodes, and the bio impedance obtaining unit may be further configured to apply a constant current to two electrodes among the four electrodes and measure the bio impedance through the other two electrodes among the four electrodes based on the 4-point measuring method.

The plurality of electrodes may include four electrodes; and the bio impedance obtaining unit may include a first switch configured to connect a first current electrode and a first voltage electrode among the four electrodes, and a second switch configured to connect a second current electrode and a second voltage electrode of the four electrodes, and may measure the bio impedance based on the 2-point measuring method.

The body weight measuring apparatus may be a device wearable on a wrist of the user.

The bio impedance obtaining unit may include four electrodes and two of the four electrodes may be arranged on an internal side of the wearable device and the other two electrodes may be arranged on an external side of the wearable device.

According to the exemplary embodiment, since the body weight measuring apparatus may measure the body weight in accordance with the bio impedance and the body information of the user regardless of the measurement pose of the user, a degree of freedom of the measurement pose may be improved.

Furthermore, since the body weight measuring apparatus according to the exemplary embodiment may be mounted in a wearable device, a degree of freedom of mounting is high so that convenience of the user to be inspected may increase.

### BRIEF DESCRIPTION OF THE DRAWINGS

The above and/or other aspects will be more apparent by describing certain exemplary embodiments, with reference to the accompanying drawings, in which:

FIGS. 1A and 1B are perspective views illustrating external appearances of a body weight measuring apparatus according to an exemplary embodiment, which respectively illustrate an external surface and an internal surface of straps;

FIG. 2 illustrates an example of a pose of a user to measure a body weight by using the body weight measuring apparatus according to the exemplary embodiment;

FIG. 3A is a block diagram illustrating a schematic configuration of a body weight measuring apparatus according to an exemplary embodiment;

FIG. 3B is a block diagram illustrating an exemplary configuration of a measuring unit included in the body weight measuring apparatus;

FIG. 4A is a circuit diagram during a measurement of a bio impedance of the user in the measurement pose of FIG. 2;

FIG. 4B is an equivalent circuit diagram of the circuit diagram of FIG. 4A;

FIG. 5A is a circuit diagram during a measurement of a bio impedance by a 4-point measuring method according to an exemplary embodiment;

FIG. 5B is a circuit diagram during a measurement of a bio impedance by a 2-point measuring method according to an exemplary embodiment;

FIG. 6 is a flowchart schematically illustrating a body weight measuring method according to an exemplary embodiment;

FIG. 7 is a flowchart schematically illustrating a method of measuring a body weight of a body by using body information of the body and measured bio impedance according to an exemplary embodiment;

FIG. 8 is a flowchart schematically illustrating a method of measuring a body weight of a body by using body information of the body and measured bio impedance according to an exemplary embodiment; and

FIG. 9 is a flowchart schematically illustrating a method of measuring a body weight of a body by using body information of the body and measured bio impedance according to an exemplary embodiment.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

It will be understood that when a layer is referred to as being “on” another layer or substrate, it can be directly on the other layer or substrate, or intervening layers may also be present.

Although the terms “first”, “second”, etc., may be used herein to describe various elements, these elements should not be limited by these terms. These terms are only used to distinguish one element from another element.

Unless otherwise defined, a singular expression may represent a plural expression. In addition, terms such as “include” and “have” are for representing that characteristics or elements described in the specification exist. It may be interpreted that one or more other characteristics or elements may be added.

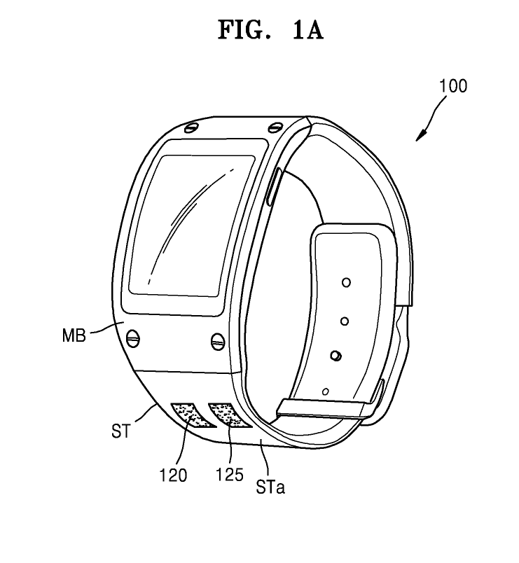

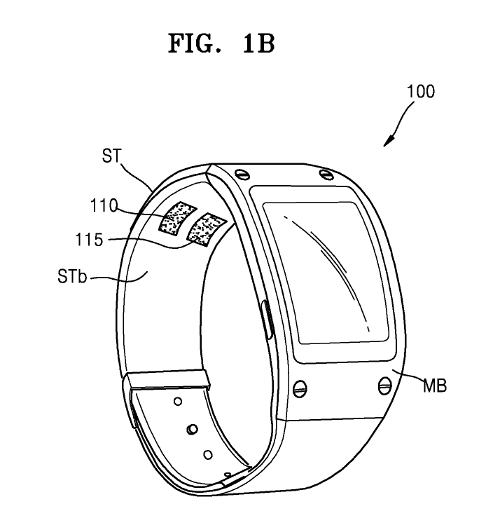

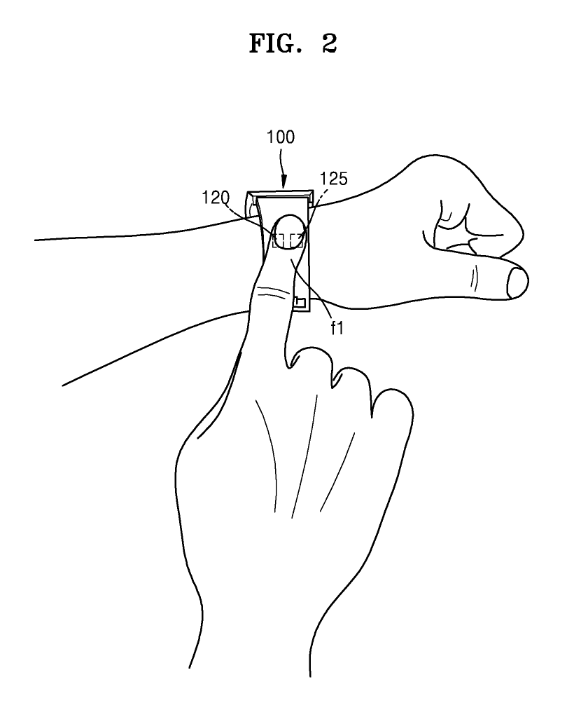

FIGS. 1A and 1B are perspective views illustrating external appearances of a wearable body weight measuring apparatus 100 that may be worn on the wrist, according to an exemplary embodiment, which respectively illustrate an external surface and an internal surface of straps. FIG. 2 illustrates an example of a pose of a user whose body weight is measured by using the body weight measuring apparatus 100 according to the exemplary embodiment.

Referring to FIGS. 1A, 1B, and 2, the body weight measuring apparatus 100 includes a main body MB and straps ST. The two straps ST may be provided on both sides of the main body MB and are connected to the main body MB, and thus, may be worn on the wrist of a body to be inspected. However, the exemplary embodiment is not limited thereto. A band-type strap ST may be integrally formed so that the main body MB is connected to both ends of the strap ST.

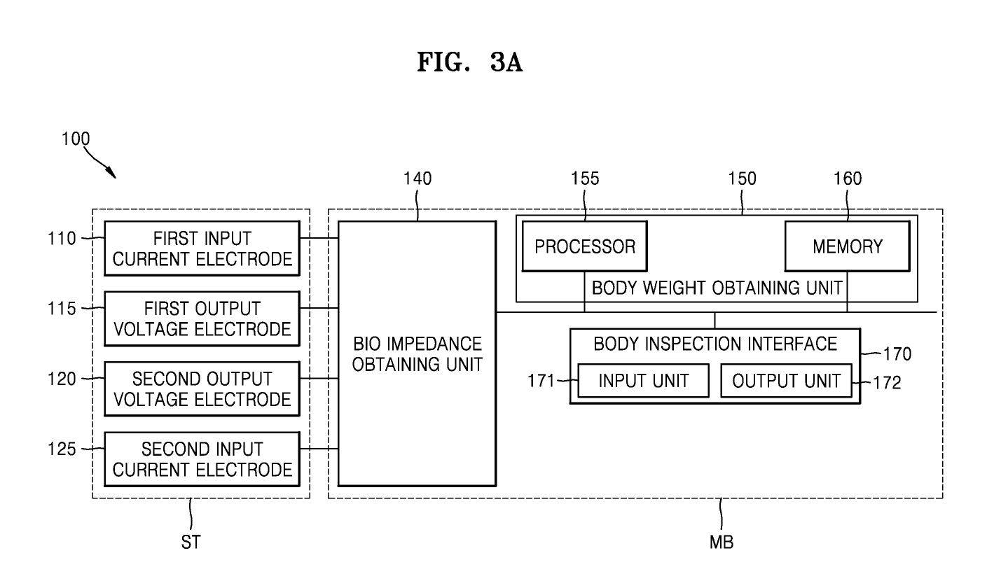

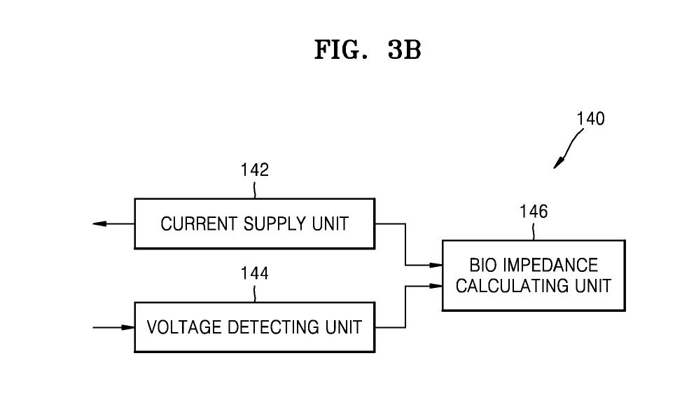

The main body MB may include body inspection interface 170 having an input unit 171 (refer to FIG. 3A) and an output unit 172 (refer to FIG. 3A). The input unit 171 of body inspection interface 170 may include a button, a keypad, a switch, a dial, or a touch interface for the body to be inspected to input body information of the body and manipulate the body weight measuring apparatus 100. The output unit 172 of body inspection interface 170 may include a display for displaying an image and may be implemented by a touch screen. The display may include a liquid crystal display (LCD) panel or an organic light-emitting diode (OLED) panel as a display panel and may display information on an analyzed body weight result as an image or text. In addition, body inspection interface 170 may include an input/output (I/O) port for connecting human interface devices (HID) with an I/O port for inputting/outputting the image.

For example, the user may input body information thereof, for example, a height, an age, and a gender thereof through the input unit 171 of the body inspection interface 170. However, the body information that may be input by the user is not limited to the height, the age, and the gender of the body to be inspected and other body information items that may represent the body information of the body to be inspected may be input. Furthermore, not all of the height, the age, and the gender of the body to be inspected have to be input and only one or more body information items among the height, the age, and the gender of the body to be inspected may be input in accordance with a selection mode.

A plurality of electrodes for measuring bio impedance may be arranged in the straps ST. The number of electrodes may be determined in accordance with a method of measuring the bio impedance. For example, when the bio impedance is measured by a 4-point measuring method, a first input current electrode 110 and a first output voltage electrode 115 may be formed on one internal surface STb of the two straps ST and a second output voltage electrode 120 and a second input current electrode 125 may be formed on one external surface STa of the two straps ST.

The first input current electrode 110 and the first output voltage electrode 115 become in contact with the wrist of the user when the user wears the body weight measuring apparatus 100.

The second output voltage electrode 120 and the second input current electrode 125 come into contact with an extremity of the other wrist of the user on which the body weight measuring apparatus 100 is not worn. The extremity of the other wrist that may contact the second output voltage electrode 120 and the second input current electrode 125 is not limited to a specific region. For example, a finger, fingers, a palm of a hand, the back of a hand, or the side of a hand may contact the second output voltage electrode 120 and the second input current electrode 125. For measurement, as illustrated in FIG. 2, a finger may simultaneously contact the second output voltage electrode 120 and the second input current electrode 125 or different fingers may respectively contact the second output voltage electrode 120 and the second input current electrode 125.

For example, the first input current electrode 110 and the first output voltage electrode 115 are illustrated as facing the second output voltage electrode 120 and the second input current electrode 125. The first input current electrode 110 and the first output voltage electrode 115 may not exactly face the second output voltage electrode 120 and the second input current electrode 125.

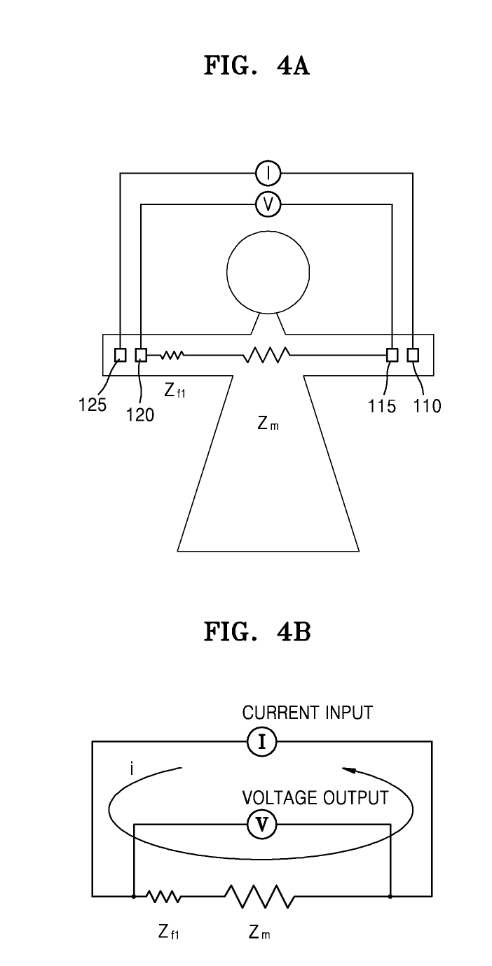

The first input current electrode 110 and the first output voltage electrode 115 may be arranged on the internal surface STb of the strap ST or an internal surface of the main body MB so as to be directly in contact with the body of the user when the user wears the weight body measuring apparatus 100. The second output voltage electrode 120 and the second input current electrode 125 may be arranged on the external surface STa of the strap ST or the external surface of the main body MB. In this case, the first input current electrode 110 and the first output voltage electrode 115 arranged on the internal surface STb of the strap ST or the internal surface of the main body MB and the second output voltage electrode 120 and the second input current electrode 125 arranged on the external surface STa of the strap ST or the external surface of the main body MB may be arranged not to run parallel with a longitudinal direction of the strap ST. For example, as illustrated in FIGS. 1A, 1B, and 2, the first input current electrode 110 and the first output voltage electrode 115 may be arranged on the internal surface STb of the strap ST and the second output voltage electrode 120 and the second input current electrode 125 may be arranged on the external surface STa of the strap ST. The first input current electrode 110 and the first output voltage electrode 115 and the second output voltage electrode 120 and the second input current electrode 125 may be arranged to be perpendicular to the longitudinal direction of the strap ST. The first input current electrode 110, the first output voltage electrode 115, the second output voltage electrode 120, and the second input current electrode 125 may form a closed circuit through both arms. The bio impedance may be measured by distinguishing a length of a path of a closed circuit that may be formed by arranging a pair of electrodes closer to a hand than the other pair of electrodes from a length of a path of a closed circuit that may be formed by arranging a pair of electrodes closer to the body than the other pair of electrodes. The method of measuring the bio impedance by using the plurality of electrodes and an arrangement relationship between the first input current electrode 110 and the first output voltage electrode 115 and the second output voltage electrode 120 and the second input current electrode 125 is described below with reference to FIGS. 3A, 3B, 4A, and 4B.

FIG. 3A is a block diagram illustrating a schematic configuration of a body weight measuring apparatus 100 according to an exemplary embodiment. FIG. 3B is a block diagram illustrating an exemplary configuration of a bio impedance obtaining unit 140 included in the body weight measuring apparatus 100. FIG. 4A is a circuit diagram during a measurement of a bio impedance of the user in the measurement pose of FIG. 2. FIG. 4B is an equivalent circuit diagram during measuring of the bio impedance in the measurement pose of FIG. 2.

Referring to FIGS. 3A and 3B, the bio impedance obtaining unit 140 applies a current to the first input current electrode 110 and the second output voltage electrode 120, measures a voltage from the first output voltage electrode 115 and the second input current electrode 125, and measures the bio impedance of the body to be inspected.

The bio impedance obtaining unit 140 may include a current supply unit 142 that may supply current to the first input current electrode 110 and the second output voltage electrode 120, a voltage detecting unit 144 that may detect the voltage between the first output voltage electrode 115 and the second input current electrode 125, and a bio impedance calculating unit 146 that may calculate the bio impedance of the body to be inspected by using the input current and the detected voltage. The voltage detecting unit 144 may include an operation amplifier that may amplify the voltage between the first output voltage electrode 115 and the second input current electrode 125 and a filter that may remove noise.

The bio impedance measured by the bio impedance obtaining unit 140 may be used by a body weight obtaining unit 150 to analyze the body weight of the user to be inspected. The body weight obtaining unit 150 may include a memory 160 that may store body information input by the user and a processor 155 that may calculate the body weight of the user by using the bio impedance measured by the bio impedance obtaining unit 140 and the body information of the user.

The processor 155 may be hardware that controls all functions and operations of the body weight measuring apparatus 100. The processor 155 executes a program stored in the memory 160 and may analyze the body weight by using the bio impedance measured by the bio impedance obtaining unit 140. A method of analyzing the body weight from the bio impedance and the body information of the body to be inspected will be described later.

The processor 155 may analyze the body weight from the bio impedance, control the bio impedance obtaining unit 140 so that the bio impedance may be measured, and process the analyzed body weight result into an image signal for display.

The processor 155 is implemented in the form of a microprocessor module or may be implemented in the form of a combination of two or more microprocessor modules. That is, the implementation form of the processor 155 is not limited.

The memory 160 may store a program and data for operating the body weight measuring apparatus 100. The memory 160 as a common storage medium may be, for example, a hard disk drive (HDD), read only memory (ROM), random access memory (RAM), a flash memory, and a memory card.

The memory 160 may store a program for correcting the bio impedance measured by the bio impedance obtaining unit 140 and a program for calculating the body weight from the corrected bio impedance and the body information of the user. In addition, the memory 160 may store data on the height, the age, and the gender of the user that are input by the user. In addition, the impedance by the extremity of the body to be inspected that is required for correcting the bio impedance, for example, finger impedance, may be stored.

In FIGS. 1A, 1B, and 3A, the first input current electrode 110, the second output voltage electrode 120, the first output voltage electrode 115, and the second input current electrode 125 are illustrated as being arranged in the strap ST and the bio impedance obtaining unit 140, the processor 155, the memory 160, and body inspection interface 170 are illustrated as being arranged in the main body MB. However, the present embodiment is not limited thereto. The first input current electrode 110, the second output voltage electrode 120, the first output voltage electrode 115, and the second input current electrode 125 may be arranged on a front surface and a rear surface of the main body MB so as to be in contact with the wrist of the user and the extremity of the other wrist on which the body weight measuring apparatus 100 is not worn. In addition, in the body weight measuring apparatus 100, a module including the first input current electrode 110, the second output voltage electrode 120, the first output voltage electrode 115, the second input current electrode 125, the bio impedance obtaining unit 140, the processor 155, the memory 160, and body inspection interface 170 may be arranged in the main body MB or the strap ST.

Referring to FIGS. 2, 4A, and 4B, the user is wearing the body weight measuring apparatus 100 on the left wrist and the right forefinger f1 of the user is in contact with the second output voltage electrode 120 and the second input current electrode 125.

In the equivalent impedance of the body to be inspected, impedances of the right arm, the body, and the left arm may form the bio impedance Zm. Resistance of the right forefinger f1 used for measurements is Zf1. Since the right forefinger f1 is in contact with both the second output voltage electrode 120 and the second input current electrode 125, as illustrated in FIGS. 4A and 4B, the impedance calculated from the voltage measured by the first output voltage electrode 115 and the second input current electrode 125 is Zm+Zf1. The bio impedance Zm is obtained by subtracting the impedance Zf1 of the right forefinger f1 from Zm+Zf1. The impedance Zf1 of the right forefinger f1 may be previously measured and may be stored in the memory 160.

During measurements, the user may use another finger such as a thumb, a middle finger, or a ring finger. In this case, the bio impedance may be calculated by correcting the measured impedance from previously stored thumb impedance, middle finger impedance, or ring finger impedance. At this time, as described above, in order for the wrist-type body weight measuring apparatus 100 to measure the bio impedance Zm, the pair of input electrodes, namely, the first input current electrode 110 and the second input current electrode 125, and the pair of output electrodes, namely, the first output voltage electrode 115 and the second output voltage electrode 120, are respectively arranged on the internal surface STb and the external surface STa of the wrist-type body weight measuring apparatus 100. According to the exemplary embodiment, the bio impedance Zm is measured by separately calculating the finger impedance Zf1 and correcting the calculated finger impedance Zf1 later. However, the present embodiment is not limited thereto. The total impedance including the finger impedance may be measured. When two or more fingers separately contact separate electrodes, the bio impedance Zm excluding the influence of the finger impedance Zf1 may be directly measured.

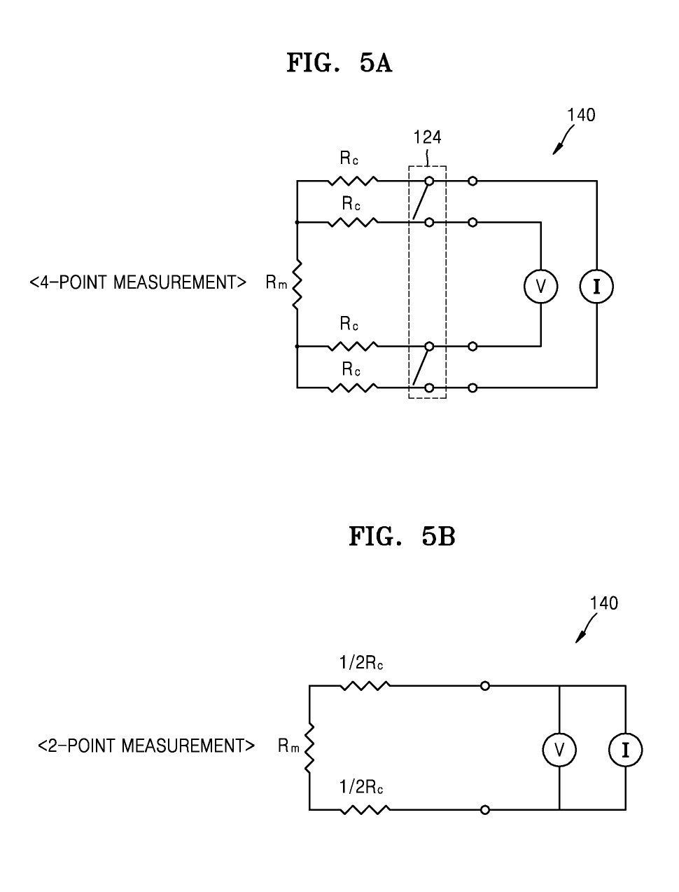

FIG. 5A is a circuit diagram during a measurement of a bio impedance by a 4-point measuring method according to an exemplary embodiment. FIG. 5B is a circuit diagram during a measurement of bio impedance by a 2-point measuring method according to an exemplary embodiment.

The method of measuring the bio impedance Zm may include the 4-point measuring method and the 2-point measuring method in accordance with the number of electrodes provided in the body weight measuring apparatus 100. Referring to FIGS. 5A and 5B, R_{C} with respect to each of the electrodes denotes contact resistance and SW denotes switches 124. The switches 124 may electrically switch the four electrodes into two input and output electrodes, for example, a first input and output electrode and a second input and output electrode. The bio impedance obtaining unit 140 may include two switches 124. The two switches 124 connect a terminal to which a current is applied and a terminal that measures a voltage. Therefore, when the switches 124 are closed, the four electrodes may electrically correspond to the two input and output electrodes.

When the switches 124 are operated so that the four electrodes are electrically switched into the two input and output electrodes, the bio impedance obtaining unit 140 may connect a current electrode and a voltage electrode among the four electrodes. A constant current is applied to the current electrode in the 4-point measuring method and the voltage electrode is connected to a voltmeter in the 4-point measuring method. The electrodes are connected and may electrically form an electrode. Areas of the respective electrodes increase double and R_{C} that denotes the contact resistance in the 4-point measuring method may become ½R_{C} in the circuit diagram illustrating the 2-point measuring method. The bio impedance obtaining unit 140 may measure the bio impedance Zm by using the connected electrodes in the 2-point measuring method. However, the present embodiment is not limited thereto. The two electrodes that may be used as the current electrode and the voltage electrode may be provided and the 2-point measuring method may be performed by using the two electrodes.

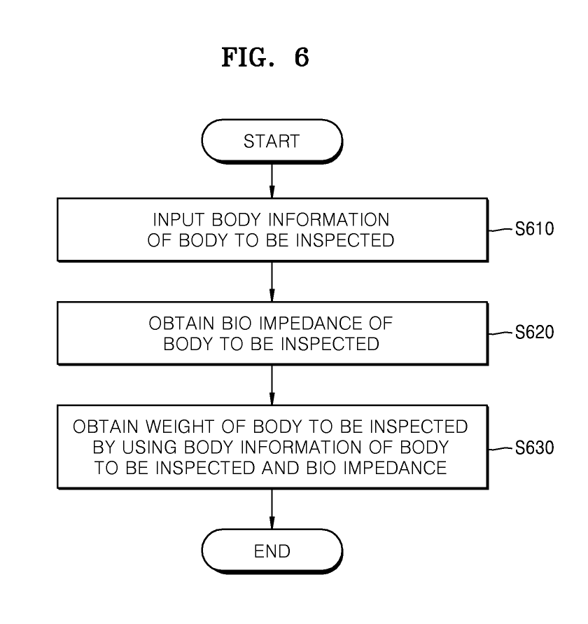

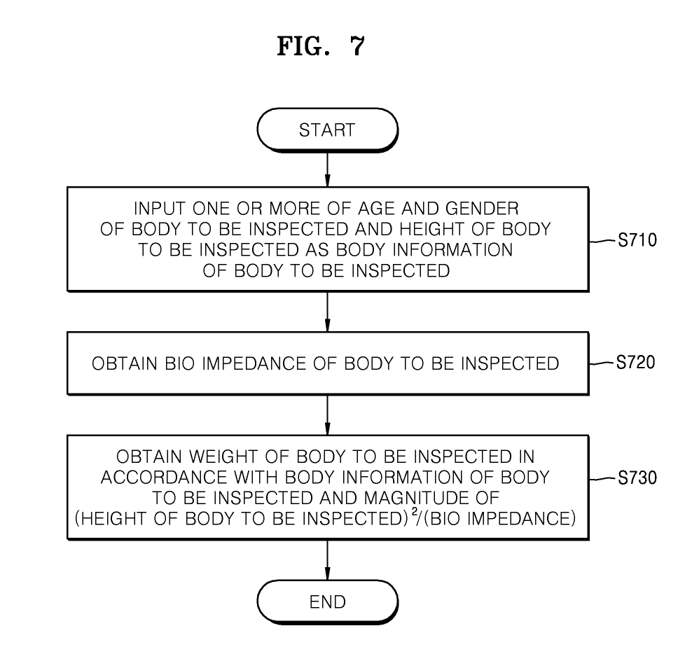

FIG. 6 is a flowchart schematically illustrating a body weight measuring method according to an exemplary embodiment. FIGS. 7 to 9 are flowcharts schematically illustrating a method of measuring a body weight of a body to be inspected by using body information of the body to be inspected and measured bio impedance according to an exemplary embodiment.

The processor 155 may obtain the weight W of the user to be inspected by using the bio impedance Zm obtained by the bio impedance obtaining unit 140 and the body information of the user which is input by the user through the input unit 171. For example, the weight W of the user may be calculated by an interaction formula using the bio impedance Zm and the body information of the user, for example, the age, the gender, and the height of the user, as variables.

INTERACTION FORMULA 1 including the age, the height, and the gender of the user or (the height of the user)̂2/(the bio impedance) as variables is as follows:

$$[ INTERACTION FORMULA 1 ]$$

$$\begin{matrix}W = {f}_{1} ( age , height , gender , {( height )}^{2} / ( Zm ) ) \\ = {a}_{0} + {a}_{1} age + {a}_{2} height + {a}_{3} gender + {{a}_{4} ( height )}^{2} / ( Zm )\end{matrix}$$

wherein, a₀, a₁, a₂, a₃, and a₄ are coefficients that may vary depending on a type of the body information input by the user. The coefficients may be previously calculated by analyzing and linearizing a plurality of data items on the INTERACTION FORMULA 1 and may be stored in the memory 160.

TABLE 1

| | a₀ | a₁ | a₂ | a₃ | a₄ |
|---|---|---|---|---|---|
| 1 | −14.192 | −0.412 | 0.358 | 4.975 | 1.356 |
| 2 | −40.258 | −0.456 | 0.527 | 0 | 1.447 |
| 3 | −54.826 | 0 | 0.609 | 6.363 | 0.715 |
| 4 | −94.836 | 0 | −94.836 | 0 | 0.745 |

Processes of calculating the weight W of the user will be described with reference to FIGS. 6 and 7, the INTERACTION FORMULA 1, and the TABLE 1. First, the user may input the body information through the input unit 171. At this time, the user may input the body information in accordance with a previously set type in operations S610, S710, S810, and S910. For example, the body information may be the height, the age, or the gender of the user. The type of the body information is not limited thereto. When an interaction formula that may process another type of body information is provided, the other type of body information may be additionally input.

After the body information is input, the bio impedance Zm of the body to be inspected may be measured by the above-described method in operations S620, S720, S820, and S920. At this time, the user may input the body information to the bio impedance obtaining unit 140 by using the 4-point measuring method or the 2-point measuring method as the method of measuring the bio impedance Zm.

When the body information is input and the bio impedance Zm of the body is measured, a coefficient corresponding to the INTERACTION FORMULA 1 is determined in accordance with the type of the input body information so that the weight W of the user may be obtained in operation S630. For example, referring to the TABLE 1, when all of the information items on the age, the gender, and the height of the user are input among the body information items, a coefficient corresponding to a first column in the TABLE 1 is applied to the INTERACTION FORMULA 1 so that the weight W of the user may be calculated. In addition, when information items on one or more of the age, the gender, and the height of the user are input among the body information items, coefficients corresponding to a second column and a third column in the TABLE 1 are applied to the INTERACTION FORMULA 1 so that the weight W of the user may be calculated. Furthermore, when only the height information is input among the body information items, a coefficient corresponding to a fourth column in the TABLE 1 is applied to the INTERACTION FORMULA 1 so that the weight W may be calculated in operation S730.

According to another exemplary embodiment, the weight W may be calculated by an interaction formula using the bio impedance Zm and the body information, for example, the age, the gender, and the height of the user as variables.

INTERACTION FORMULA 2 including the age, the height, and the gender of the user or (the height of the user)/(the bio impedance) as variables is as follows:

$$[ INTERACTION FORMULA 2 ]$$

$$\begin{matrix}W = {f}_{2} ( age , height , gender , ( height ) / ( Zm ) ) \\ = {a}_{0} + {a}_{1} age + {a}_{2} height + {a}_{3} gender + {a}_{4} ( height ) / ( Zm )\end{matrix}$$

wherein, a₀, a₁, a₂, a₃, and a₄ are coefficients that may vary depending on a type of the body information input by the user to be inspected. The coefficients may be previously calculated by analyzing and linearizing a plurality of data items on the INTERACTION FORMULA 2 and may be stored in the memory 160.

TABLE 2

| | a₀ | a₁ | a₂ | a₃ | a₄ |
|---|---|---|---|---|---|
| 1 | −50.875 | −0.402 | 0.576 | 4.812 | 227.16 |
| 2 | −77.571 | −0.447 | 0.747 | 0 | 244.62 |
| 3 | −74.521 | 0 | 0.727 | 6.267 | 118.60 |
| 4 | −114.06 | 0 | 0.98 | 0 | 125.78 |

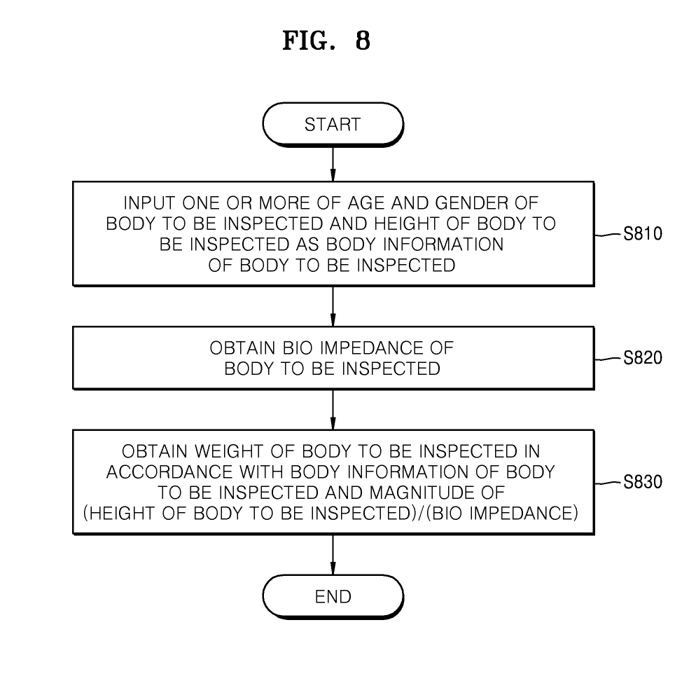

Referring to FIGS. 6 and 8, the INTERACTION FORMULA 2, and the TABLE 2, when the body information of the user is input and the bio impedance Zm of the user is measured, a coefficient corresponding to the INTERACTION FORMULA 2 is determined in accordance with the type of the input body information so that the weight W of the user may be obtained in operation S630. For example, referring to the TABLE 2, when all of the information items on the age, the gender, and the height of the user are input among the body information items of the user, a coefficient corresponding to a first column in the TABLE 2 is applied to the INTERACTION FORMULA 2 so that the weight W of the user may be calculated. In addition, when information items on one or more of the age, the gender, and the height of the user are input among the body information items, coefficients corresponding to a second column and a third column in the TABLE 2 are applied to the INTERACTION FORMULA 2 so that the weight W may be calculated. Furthermore, when only the height information is input among the body information items, a coefficient corresponding to a fourth column in the TABLE 2 is applied to the INTERACTION FORMULA 2 so that the weight W may be calculated in operation S830.

According to another exemplary embodiment, the weight W of the user may be calculated by an interaction formula using the bio impedance Zm and the body information, for example, the age, the gender, and the height of the user, as variables.

INTERACTION FORMULA 3 including the age, the height, and the gender of the user or the bio impedance Zm as variables is as follows:

$$[ INTERACTION FORMULA 3 ]$$

$$\begin{matrix}W = {f}_{3} ( age , height , gender , Zm ) \\ = {a}_{0} + {a}_{1} age + {a}_{2} height + {a}_{3} gender + {a}_{4} Zm\end{matrix}$$

wherein, a₀, a₁, a₂, a₃, and a₄ are coefficients that may vary depending on a type of the body information input by the user. The coefficients may be previously calculated by analyzing and linearizing a plurality of data items on the INTERACTION FORMULA 3 and may be stored in the memory 160.

TABLE 3

| | a₀ | a₁ | a₂ | a₃ | a₄ |
|---|---|---|---|---|---|
| 1 | −6.3485 | −0.390 | 0.713 | 5.210 | −0.30 |
| 2 | −34.705 | −0.431 | 0.920 | 0 | −0.310 |
| 3 | −45.913 | 0 | 0.780 | 6.439 | −0.017 |
| 4 | −87.006 | 0 | 1.051 | 0 | −0.017 |

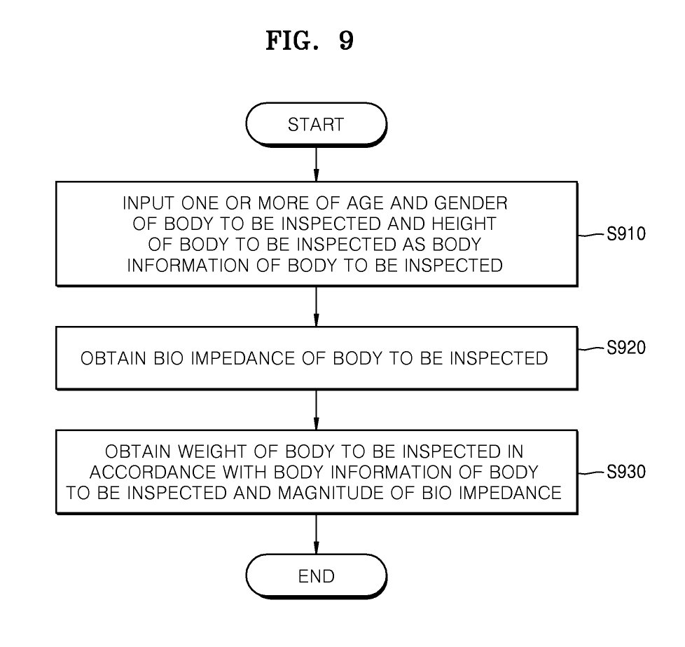

Referring to FIGS. 6 and 9, the INTERACTION FORMULA 3, and the TABLE 3, when the body information of the user to be inspected is input and the bio impedance Zm of the user is measured, a coefficient corresponding to the INTERACTION FORMULA 3 is determined in accordance with the type of the input body information so that the weight W of the user may be obtained in operation S630. For example, referring to the TABLE 3, when all of the information items on the age, the gender, and the height of the user are input among the body information items of the user, a coefficient corresponding to a first column in the TABLE 3 is applied to the INTERACTION FORMULA 3 so that the weight W of the user may be calculated. In addition, when information items on one or more of the age, the gender, and the height of the user are input among the body information items, coefficients corresponding to a second column and a third column in the TABLE 3 are applied to the INTERACTION FORMULA 3 so that the weight W may be calculated. Furthermore, when only the height information is input among the body information items, a coefficient corresponding to a fourth column in the TABLE 3 is applied to the INTERACTION FORMULA 3 so that the weight W may be calculated in operation S930.

As described above, according to the exemplary embodiment, since the body weight measuring apparatus may measure the body weight in accordance with the bio impedance and the body information regardless of the measurement pose of the body to be inspected, a degree of freedom of the measurement pose is high so that convenience of the body to be inspected may increase. Furthermore, since the body weight measuring apparatus according to the exemplary embodiment may be mounted in a wearable device, a degree of freedom of mounting is high so that convenience of the body to be inspected may increase.

While not restricted thereto, an exemplary embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an exemplary embodiment may be written as a computer program transmitted over a computer-readable transmission medium, such as a carrier wave, and received and implemented in general-use or special-purpose digital computers that execute the programs. Moreover, it is understood that in exemplary embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A body weight measuring method performed by a body weight measuring apparatus, the body weight measuring method comprising:
receiving body information of a user other than bio impedance information by an input unit of the apparatus;
measuring bio impedance of the user by a bio impedance obtaining unit of the apparatus; and
obtaining, by a processor of the apparatus, a weight of the user based on the measured bio impedance and the received body information.

2. The body weight measuring method of claim 1, wherein the bio information comprises a height of the user, and
the obtaining the weight of the user comprises calculating the weight in accordance with the body information and a magnitude of (the height of the user)²/(the bio impedance).

3. The body weight measuring method of claim 2, wherein the body information further comprises a height of the user, and at least one of an age and a gender.

4. The body weight measuring method of claim 1, wherein the bio information comprises a height of the user, and
the obtaining the weight of the user comprises calculating the weight in accordance with the body information and a magnitude of (the height of the user)/(the bio impedance).

5. The body weight measuring method of claim 4, wherein the body information further comprises a height of the user, and at least one of an age and a gender.

6. The body weight measuring method of claim 1, wherein the obtaining the weight of the user comprises calculating the weight in accordance with the body information and a magnitude of the bio impedance.

7. The body weight measuring method of claim 6, wherein the body information comprises at least one of an age, a gender, and a height of the user.

8. The body weight measuring method of claim 1, wherein the measuring the bio impedance comprises measuring the bio impedance based on a 4-point measuring method or a 2-point measuring method.

9. The body weight measuring method of claim 8, wherein the measuring the bio impedance based on the 4-point measuring method comprises applying a constant current to two electrodes among four electrodes of the apparatus and measuring the bio impedance through the other two electrodes among the four electrodes.

10. The body weight measuring method of claim 8, wherein the measuring the bio impedance based on the 2-point measuring method comprises connecting first and second electrodes of four electrodes of the apparatus so that the first and second electrodes are switched into an input electrode of the apparatus, and connecting third and fourth electrodes of the four electrodes so that the third and fourth electrodes are switched into an output electrode of the apparatus.

11. A non-transitory computer-readable storage medium storing a program that is executable by a computer to perform the method of claim 1.

12. A body weight measuring apparatus comprising:
an input unit configured to receive body information of a user other than bio impedance information;
a bio impedance obtaining unit comprising a plurality of electrodes configured to be in contact with the user to measure a bio impedance of the user; and
a body weight obtaining unit configured to obtain a weight of the user based on the measured bio impedance and the received body information.

13. The body weight measuring apparatus of claim 12, further comprising:
a memory configured to store a program that calculates the weight of the user; and
a processor configured to load the program and calculate the weight of the user in accordance with the body information and a magnitude of the bio impedance by using the program.

14. The body weight measuring apparatus of claim 12, wherein the bio impedance obtaining unit is further configured to measure the bio impedance based on a 4-point measuring method or a 2-point measuring method.

15. The body weight measuring apparatus of claim 14, wherein the plurality of electrodes comprise four electrodes, and
the bio impedance obtaining unit is further configured to apply a constant current to two electrodes among the four electrodes and measure the bio impedance through the other two electrodes among the four electrodes based on the 4-point measuring method.

16. The body weight measuring apparatus of claim 14, wherein the plurality of electrodes comprise four electrodes, and
wherein the bio impedance obtaining unit comprises a first switch configured to connect a first current electrode and a first voltage electrode among the four electrodes, and a second switch configured to connect a second current electrode and a second voltage electrode of the four electrodes, and measure the bio impedance based on the 2-point measuring method.

17. The body weight measuring apparatus of claim 12, wherein the body weight measuring apparatus is a device wearable on a wrist of the user.

18. The body weight measuring apparatus of claim 17, wherein the bio impedance obtaining unit comprises four electrodes, and
wherein two of the four electrodes are arranged on an internal side of the wearable device and the other two electrodes are arranged on an external side of the wearable device.
